(19) United States
(12) Patent Application Publication
(10) Pub. No.: US 2023/0129262 A1
(43) Pub. Date: Apr. 27, 2023

(54) METHODS AND PHARMACEUTICAL COMPOSITIONS OF THROMBOXANE A2 RECEPTOR ANTAGONIST FOR THE TREATMENT OF COVID-19

(71) Applicant: Martin OGLETREE, Bala Cynwyd, PA (US)

(72) Inventor: Martin OGLETREE, Bala Cynwyd, PA (US)

(21) Appl. No.: 17/915,608

(22) PCT Filed: Mar. 30, 2021
(86) PCT No.: PCT/US21/24760

§ 371 Date: Sep. 29, 2022

Related U.S. Application Data
(60) Provisional application No. 63/002,511, filed on Mar. 31, 2020.

(51) Int. Cl. A61K 31/422; A61K 45/06

(52) U.S. Cl. CPC A61K 31/422; A61K 45/06

(57) ABSTRACT

The present invention is related to the use of thromboxane A2 receptor antagonists (e.g., 3-[2-[[(1S,2R,3S,4R)-3-[4-(pentylcarbamoyl)-1,3-oxazol-2-yl]-7-oxabicyclo[2.2.1]heptan-2-yl]methyl]phenyl]propanoic acid (Ifetroban), or one, or a mixture of more than one pharmaceutically acceptable salts thereof) in the treatment of SARS-CoV-2 infection in humans, and pharmaceutical compositions for the same comprising thromboxane A2 receptor antagonists (e.g., ifetroban) in an effective amount to treat and/or prevent conditions resulting from such infection.

### FIELD OF THE INVENTION

The present invention is related to the use of thromboxane A2 receptor antagonists (e.g., Ifetroban) and pharmaceutical compositions thereof in an effective amount for the treatment of SARS-CoV-2 infection (e.g., COVID-19) in mammals (e.g., humans) to treat this disease.

### BACKGROUND OF THE INVENTION

Coronavirus disease 2019 (COVID-19) is a highly contagious, severe acute respiratory syndrome (SARS) caused by the coronavirus, SARS-CoV-2. Upon human exposure, the coronavirus gains entry into cells of the respiratory tract primarily following attachment to angiotensin converting enzyme 2 (ACE2) on cell membranes of lung type II alveolar cells, arterial and venous endothelial cells, and arterial smooth muscle cells. Coronavirus replication, cellular disruption and spread to other organs (e.g., heart, brain, kidneys, intestines) lead to symptomatic COVID-19. Underlying diseases (e.g., obesity, hypertension, diabetes) and impaired host defense render certain populations more vulnerable to severe disease (e.g., elderly with comorbidities). From March, 2020 through February, 2021, COVID-19 caused more than 500,000 deaths in the United States alone.

Early symptoms of COVID-19 include pulmonary dysfunction (e.g., cough, shortness of breath, hypoxemia), fever, fatigue, muscle aches, headache, loss of taste or smell, nausea, and diarrhea. Some people rapidly progress from difficulty breathing to respiratory failure (e.g., needing mechanical ventilation), heart failure, intensive care unit treatment, and often death. Complications of COVID-19 may include pneumonia, pulmonary edema, acute respiratory distress syndrome (ARDS), pulmonary fibrosis, blood clots, cardiomyopathy, heart failure, acute kidney injury, and neurological issues like brain fog. Serious illness with COVID-19 is typically seen with people older than 60 years of age, but younger people are more at-risk to develop arterial thrombosis including myocardial infarction and ischemic stroke. Recovery from COVID-19 may take many months, and so-called long-haulers may suffer prolonged symptoms including cough, dyspnea, fatigue, body aches, joint pain, loss of taste and smell, difficulty sleeping, headaches, and brain fog. (Marshall 2020) Persistent health problems in some patients may be attributable to development of pulmonary fibrosis. (McDonald 2021)

Authorized or approved treatments for COVID-19 in hospitalized patients currently target inhibiting viral replication (e.g., remdesivir), suppressing inflammation (e.g., dexamethasone), and enhancing the immune response (e.g., convalescent plasma and monoclonal antibodies). Vaccines are now available and being administered to millions of people to prevent disease progression following SARS-CoV-2 exposure. However, variants of the original SARS-CoV-2 are emerging and may be resistant to certain antibodies and vaccines. Such variants may become dominant strains that may be more transmissible and/or virulent. Moreover, COVID-19 remains a contagious disease with inadequately effective treatment options.

Pulmonary hypertension is increased blood pressure in the pulmonary circulation that may be attributable to pulmonary arterial constriction, pulmonary vascular obstruction (e.g., with clots, thrombi, inflammatory cells, or emboli), pulmonary venous constriction, which raises pulmonary capillary pressure due to post-capillary pulmonary vasoconstriction, or downstream obstruction of blood flow (e.g., mitral valve stenosis). (Ganter, Jakob et al. 2006) An invasive assessment using right heart catheterization concluded that post-capillary pulmonary hypertension was present in 76% of COVID-19 patients. Pulmonary artery wedge pressure was higher in COVID-19 than in ARDS patients and inversely related to lung compliance. (Caravita, Baratto et al. 2020) Post-capillary pulmonary vasoconstriction, elevated pulmonary capillary pressures, and higher pulmonary artery wedge pressures force vascular fluid into airways, leading to pulmonary edema.

Pulmonary edema is fluid accumulation in terminal airways (e.g., alveoli) where it interferes with normal gas exchange (e.g., oxygenation of blood), reduces lung compliance, and produces difficulty breathing. Pulmonary edema may be caused by increased pulmonary microvascular hydrostatic pressure and/or increased microvascular permeability, and edema formation is exaggerated by combined increases in capillary pressure and permeability. Permeability may increase following vascular endothelial injury, formation of inter-endothelial cell gaps, and/or disruption of the endothelial glycocalyx lining the vascular lumen. In COVID-19 pneumonia, areas of pulmonary consolidation (i.e., normally compressible lung tissue that has filled with liquid instead of air) are more radio-opaque than normally aerated lung parenchyma and are clearly visible in radiography and on computed tomography (CT) scans. Pulmonary edema is a hallmark of ARDS in which lung capillary permeability is elevated. In COVID-19, both lung capillary pressure and permeability may be elevated leading to exaggerated pulmonary edema.

Respiratory failure is impaired pulmonary gas exchange leading to inadequate oxygenation of blood that may be attributable to pulmonary dysfunction, lung injury and/or ventilation-perfusion mismatching. Providing supplemental oxygen is required but may not be adequate to normalize blood oxygen saturation, which leads to tracheal intubation and mechanical ventilation. Patients seriously ill with COVID-19 may progress from shortness of breath and hypoxemia to ARDS-like respiratory failure with mortality of 39%. (Hasan, Capstick et al. 2020)

Fibrosis is the formation of excess fibrous connective tissue (e.g., collagen) in an organ or tissue in a reparative or reactive process. Formation of fibrous tissue is a physiological process, and fibrous tissue is a normal constituent of organs or tissues in the body. Normally, fibrous connective tissue is deposited at sites of injury as part of the wound healing process, and this can lead to temporary or permanent scarring. Fibroblasts are the effector cells in fibrosis and are found in every tissue in the body, providing structural support and a scaffold for tissue repair following injury. In pathological fibrosis, myofibroblasts produce dense fibrous connective tissue in a fibroproliferative response to injury and/or triggering signals. Resulting scars may permanently damage the structure and functions of the affected tissue as in liver cirrhosis or pulmonary fibrosis. Pulmonary fibrosis is excess deposition of fibrin, extracellular matrix, connective tissue, and scarring of the lung. Scarring may alter lung structure, displace functional tissue, and contribute to pulmonary dysfunction. Clinical, radiographic, and autopsy data indicate that pulmonary fibrosis develop in severe acute respiratory distress syndrome (SARS) pathology, and current evidence suggests that pulmonary fibrosis also complicates COVID-19. Following SARS-CoV-2 infection, damage to terminal airways and pulmonary vasculature elicits fibrosis in response to lung injury. Fibrotic lung scarring can be seen with medical imaging (i.e., high resolution computed tomography scans) in COVID-19 patients during hospitalization and convalescence. On autopsy, patients who died of COVID-19 pneumonia show features of diffuse alveolar damage with areas of pulmonary consolidation with fluid accumulation, fibroproliferation, and deposition of extracellular matrix and fibrin in the alveolar space. (Ojo, Balogun et al. 2020)

Thrombosis is obstruction of blood vessels with aggregates of platelets, coagulated blood clots or both that may arise from a local response to vascular injury, disease, prothrombotic factors and/or blood flow stasis. Hemostasis is the physiological response to vascular injury where adhesion of platelets, blood coagulation, and fibrin deposition limits blood loss and bleeding. Life-threatening thrombosis is responsible for myocardial infarction (i.e., coronary artery or stent thrombosis), ischemic stroke (i.e., thrombosis in arteries supplying blood to brain tissues), and venous thromboembolism (i.e., pulmonary embolism arising from dislodged venous clots in the legs). Patients with COVID-19 typically experience arterial and/or venous thrombosis, particularly numerous platelet-rich thrombi in small arteries, termed thrombotic microangiopathy. In the pulmonary circulation this is associated with areas of diffusely edematous lung tissue. Thrombosis in COVID-19 often also affects organs in addition to the lungs including the brain, heart, liver and kidneys. (Gu, Tyagi et al. 2021) Among patients with emergent large vessel occlusion strokes, patients with COVID-19 were significantly younger than patients without COVID-19, 59±13 versus 74±17; P=0.004. (Majidi, Fifi et al. 2020) Thromboxane (Tx) A2 is a short-lived polyunsaturated fatty acid that is a product of fatty acid cyclooxygenase (COX) 1 and COX-2 metabolism and subsequent prostaglandin (PG) endoperoxide (i.e., PGH2) metabolism by TxA synthase. COX inhibitors block synthesis of both PGH2 and TxA2. TxA synthase inhibitors selectively inhibit TxA2 synthesis without inhibiting PGH2 formation. The inactive metabolite of TxA2 may be measured in blood plasma as TxB2 and urinary excretion of circulating TxA2 metabolite may be measured as 2,3-dinor-TXB2 and 11-dehydro-TXB2. TxA2 is produced primarily by activated platelets and macrophages and is a potent mediator of platelet aggregation, vasoconstriction, pulmonary venoconstriction, bronchoconstriction, vascular endothelial permeability, tissue factor expression, and other biological activities. The biosynthesis of PGH2 and TxA2 is inhibited by aspirin (acetylsalicylic acid) and other non-steroidal anti-inflammatory drugs. Low-dose aspirin (81-100 mg/day, p.o.) selectively inhibits platelet PGH2 and TxA2 synthesis. Aspirin is an effective antithrombotic agent that is indicated for secondary prevention of myocardial infarction and stroke. Aspirin also prevents platelet activation in venous thrombosis. (Tarantino, Amadio et al. 2016) COVID-19 patients exhibited elevated plasma TxB2 levels, and plasma TxB2 concentration correlated with thrombosis and all-cause mortality. The association of TxB2 with thrombosis and mortality was seen whether or not COVID-19 patients were treated with aspirin. (Barrett, Lee et al. 2020) In hospitalized COVID-19 patients, low-dose aspirin use was independently associated with decreased risk of mechanical ventilation, ICU admission, and in-hospital mortality. (Chow, Khanna et al. 2021) COVID-19 patients given high-dose aspirin (1,000 mg/day) also experienced reduced mortality compared to propensity score matched COVID-19 controls. (Liu, Huang et al. 2021)

Thromboxane-prostanoid (TP) receptors mediate the direct cellular effects of TxA2, PGH2 and certain isoprostanes. TP receptors are expressed on platelets, smooth muscle cells, endothelial cells, fibroblasts, monocytes, cardiac myocytes, glomerular mesangial cells, Kupffer cells, oligodendrocytes, afferent nerve endings, astrocytes, and immature thymocytes. (Nakahata 2008) TP receptor activation leads to platelet aggregation, selective pulmonary venoconstriction, tissue-selective vascular endothelial permeability, and tissue factor expression on endothelial cells and monocytes. Bode, Mackman 2004) Consequences of TP receptor activity may include arterial and/or venous thrombosis, pulmonary venoconstriction, pulmonary hypertension (particularly elevated pulmonary capillary pressure), lung vascular permeability, pulmonary edema, and sudden death. These TP receptor-dependent effects may be inhibited by TP receptor antagonists like ifetroban.

### BRIEF DESCRIPTION OF THE INVENTION

In accordance with the above background, the present invention provides for methods of treating COVID-19 by administering a therapeutically effective amount of a TP receptor antagonist to a patient in need thereof.

In accordance with the above background and other literature, the present invention is directed in part to a method of treating or ameliorating COVID-19 in a subject in need of treatment, comprising administering a therapeutically effective amount of a TP receptor antagonist to the patient. The COVID-19 related pulmonary capillary hypertension contributes to hypoxemia and is confirmed by measuring arterial blood oxygen saturation. The COVID-19 related pulmonary edema contributes to dyspnea and is confirmed radiologically as pulmonary consolidation. The COVID-19 related fibrosis restricts pulmonary function and is confirmed radiologically. The COVID-19 related pulmonary thrombotic microangiopathy contributes to ventilation-perfusion mismatching and is confirmed by elevated plasma fibrin D-dimer and arterial blood oxygen saturation. The TP receptor antagonist may be administered orally, intranasally, by inhalation, rectally, vaginally, sublingually, buccally, parenterally, or transdermally. In certain preferred embodiments, the method further comprises administering the TP receptor antagonist to the patient on a chronic basis. In certain embodiments, the TP receptor antagonist comprises a therapeutically effective amount of 3-[2-[[(1S,2R,3S,4R)-3-[4-(pentylcarbamoyl)-1,3-oxazol-2-yl]-7-oxabicyclo[2.2.1]heptan-2-yl]methyl]phenyl]propanoic acid (Ifetroban), and pharmaceutically acceptable salts thereof. In certain other embodiments, the TP receptor antagonist comprises a therapeutically effective amount of 3-[2-[[(1S,2R,3S,4R)-3-[4-(pentylcarbamoyl)-1,3-oxazol-2-yl]-7-oxabicyclo[2.2.1]heptan-2-yl]methyl]phenyl]propanoic acid, monosodium salt (Ifetroban Sodium). In certain preferred embodiments, the pulmonary function of the patient is maintained or improved.

Certain embodiments of the invention are directed to the method, wherein the TP receptor antagonist is administered prophylactically to prevent respiratory failure in the patient, and/or to prophylactically to prevent pulmonary edema in the patient. In certain preferred embodiments, the therapeutically effective amount is from about 10 mg to about 1,500 mg. In certain preferred embodiments, the TP receptor antagonist is Ifetroban sodium and the therapeutically effective amount is from about 50 mg to about 250 mg per day. In certain embodiments, the ifetroban is administered orally. In certain embodiments, the present invention is directed to a method of treating and/or ameliorating COVID-19 in a patient in need thereof, comprising administering to a patient in need thereof a therapeutically effective amount of a TP receptor antagonist to provide a desired plasma concentration of the TP receptor antagonist of about 0.1 ng/mL to about 10,000 ng/mL.

The invention is also directed to a method of providing relief from shortness of breath or hypoxemia for a human patient(s) suffering from COVID-19 the administration of a TP receptor antagonist as described herein.

The invention is further directed to a method of improving oxygenation of blood and oxygen delivery to tissues by reducing pulmonary edema in a human patient(s) suffering from COVID-19 via the administration of a TP receptor antagonist as described herein.

The invention is further directed to a method of improving oxygenation of blood and oxygen delivery to tissues by reducing COVID-19 related pulmonary capillary hypertension via the administration of a TP receptor antagonist as described herein.

The invention is further directed to a method of improving oxygenation of blood and oxygen delivery to tissues by reducing COVID-19 related pulmonary thrombotic microangiopathy via the administration of a TP receptor antagonist as described herein.

The invention is further directed to a method of treating pulmonary dysfunction in a human patient suffering from COVID-19, comprising chronically administering a therapeutically effective amount of a TP receptor antagonist to the human patient. In certain preferred embodiments, the thromboxane A2 receptor antagonist is 3-[2-[[(1S,2R,3S,4R)-3-[4-(pentylcarbamoyl)-1,3-oxazol-2-yl]-7-oxabicyclo[2.2.1]heptan-2-yl]methyl]phenyl]propanoic acid (Ifetroban), and pharmaceutically acceptable salts thereof, and in certain most preferred embodiments the TP receptor antagonist is 3-[2-[[(1S,2R,3S,4R)-3-[4-(pentylcarbamoyl)-1,3-oxazol-2-yl]-7-oxabicyclo[2.2.1]heptan-2-yl]methyl]phenyl]propanoic acid, monosodium salt (Ifetroban Sodium). The therapeutically effective amount may be, e.g., from about 50 mg to about 300 mg. The TP receptor antagonist may be administered, e.g., in an amount from about 50 or 100 mg to about 250 mg per day. In certain embodiments, the TP receptor antagonist is ifetroban or a pharmaceutically acceptable salt thereof and the daily dose is from about 50 mg to about 250 mg per day. In certain embodiments, the ifetroban is administered orally. In certain embodiments, the pulmonary dysfunction is pulmonary edema and lung stiffness. In certain embodiments, the therapeutically effective amount of ifetroban provides improved lung mechanics and oxygenation of blood of the patient.

The present invention also relates to methods and compositions for treating COVID-19 in a mammal(s) or human(s) in need of treatment thereof, the method comprising administering a therapeutically effective amount of a TP receptor antagonist to a subject(s) or patient(s) in need thereof. Preferably, the method of treatment comprises administering a composition comprising administering a therapeutically effective amount of a TP receptor antagonist to a COVID-19 patient in need thereof in an amount effective to improve pulmonary function. Further provided is a method of preventing pulmonary fibrosis in a subject(s) or patient(s) in need of such treatment, comprising administering a composition comprising a TP receptor antagonist in an amount effective to reduce the formation of fibrotic tissue that would occur in the absence of such treatment.

### DETAILED DESCRIPTION OF THE INVENTION

In accordance with the above stated background, it is believed that administration of a therapeutically effective amount of a TP receptor antagonist to a subject(s) or patient(s) in need thereof can treat pulmonary dysfunction associated with SARS-CoV-2 infection or COVID-19. The phrase “therapeutically effective amount” refers to that amount of a substance that produces some desired local or systemic effect at a reasonable benefit/risk ratio applicable to any treatment. The effective amount of such substance will vary depending upon the subject and disease condition being treated, the weight and age of the subject, whether the subject is fasted or fed, the severity of the disease condition, the manner of administration and the like, which can readily be determined by one of ordinary skill in the art.

The TP receptor is a G protein-coupled receptor spanning membranes located in platelets, immune cells, smooth muscle, endothelial cells, fibroblasts, and cardiomyocytes, and its sustained activation may have deleterious consequences in the lungs. For example, a gain-of-function mutation in TBXA2R, the human TP receptor gene, was identified in Phenome-Wide Association Studies (PheWAS) because this mutation associated with a higher-than-normal incidence of metastatic cancers as well as pulmonary heart disease, pulmonary hypertension, primary pulmonary hypertension, and lung transplantation. (Pulley, Jerome et al. 2018, Werfel, Hicks et al. 2020)

West has shown that blockade of the TP receptor with ifetroban dramatically decreases right ventricular fibrosis and improves cardiac function in a pressure-overload model of pulmonary arterial hypertension (West, Voss et al. 2016) and in a model of Duchenne muscular dystrophy (West, Galindo et al. 2019).

In hospitalized COVID-19 patients, synthesis of TxA2, evidenced by the plasma concentration of TxB2, is strongly correlated with mortality. (Barrett, Lee et al. 2020) Critically ill patients suffering from COVID-19 may be diagnosed with adult respiratory distress syndrome (ARDS) characterized by pulmonary edema resulting from lung injury, increased lung vascular permeability, and fluid accumulation in terminal airways. However, the cardiopulmonary dynamics in COVID-19 are subtly different from typical ARDS in the following ways: “pulmonary vascular resistance of COVID-19 patients was normal, similar to that of control subjects [1.6 (1.1-2.5) vs. 1.6 (0.9-2.0) WU, P=0.343], and lower than reported in ARDS patients (P<0.01). Pulmonary hypertension was present in 76% of COVID-19 patients and in 19% of control subjects (P<0.001), and it was always post-capillary. Pulmonary artery wedge pressure was higher in COVID-19 than in ARDS patients, and inversely related to lung compliance (r=−0.46, P=0.038).” (Caravita, Baratto et al. 2020) Note that pulmonary hypertension in COVID-19 patients is post-capillary as reflected in higher pulmonary artery wedge pressure (an estimate of pulmonary capillary blood pressure) and inversely related to lung compliance—higher lung stiffness largely due to pulmonary edema. TP receptor dependent post-capillary pulmonary hypertension can result from selective pulmonary venoconstriction which raises pulmonary artery wedge pressure. (Wakerlin, Finn et al. 1995) COVID-19 post-mortem lung tissue revealed platelet aggregates obstructing the microvasculature. (Ackermann, Verleden et al. 2020) The mediator(s) responsible for lung pathology in COVID-19 patients is unknown, but increased TxA2 synthesis and resulting platelet aggregation, pulmonary venoconstriction, and increased vascular endothelial permeability are consistent with a major causative role of TxA2 and TP receptor activation.

In SARS-CoV-2 mediated lung injury, higher pulmonary capillary pressure, due to post-capillary pulmonary hypertension, can greatly exaggerate lung fluid accumulation, overwhelm lymphatic drainage of lung water, and cause pulmonary edema. Elevated TxA2 and TP receptor activation in the pulmonary circulation are known to cause pulmonary hypertension due to selective pulmonary venoconstriction (i.e., contraction of post-capillary pulmonary venules and veins) which elevates pulmonary capillary blood pressure. (Yoshimura, Tod et al. 1989) Treatment of COVID-19 with a TP receptor antagonist like ifetroban may lower elevated pulmonary capillary pressure, reduce pulmonary edema, improve lung mechanics, shorten hospital stay and improve survival. Early treatment of SARS-CoV-2 infection with ifetroban may prevent development of post-capillary pulmonary hypertension, pulmonary edema and lung stiffness.

Elevated pulmonary capillary pressure promotes lung fluid accumulation, which may be greatly exaggerated when pulmonary vascular permeability is increased. The stable TxA2 mimetic, U-46,619 (9,11-dideoxy-9α, 11α-methanoepoxy prostaglandin F2α), activates TP receptors. In preclinical studies, U-16,619 infusion strongly increased plasma fluid and protein accumulation in the lung, and this effect was completely blocked by a TP receptor antagonist, SQ29548. Smaller TP receptor dependent increases in plasma fluid and protein accumulation were seen in the heart and kidneys. The authors concluded: “The present findings demonstrate that TxA2 receptor activation acutely increased hematocrit, probably by inducing a shift of plasma fluid from the vascular compartment toward the interstitium. This hypothesis was confirmed in studies using Evans blue dye as a reliable marker of albumin extravasation; the results demonstrate the existence of organ-specific increases in microvascular shift of albumin and possibly other proteins.” (Bertolino, Valentin et al. 1995) These effects on transvascular fluid and protein flux require not only an increase in capillary blood pressure but also increases in vascular permeability.

In patients with acute lung injury, TP receptor blockade with ifetroban reduced pulmonary capillary pressure by selectively relaxing pulmonary veins and decreasing post-capillary resistance. (Schuster, Kozlowski et al. 2001) In COVID-19 patients with coronavirus-mediated lung injury, TP receptor dependent pulmonary venoconstriction will aggravate lung fluid accumulation and exaggerate pulmonary edema, and this life-threatening disease process may be improved by TP receptor blockade with ifetroban.

Lung injury provocations trigger release of TxA2, and inhibition of TxA2 synthesis or activity ameliorates many but not all these early lung injury responses (e.g., pulmonary hypertension, hypoxemia, pulmonary edema). In particular, TP receptor blockade with ifetroban (also known as SQ34451 and BMS-180291) or closely related 7-oxabicyclo[2.2.1] heptane compounds (i.e., SQ29548, SQ28668 and SQ30741) inhibited lung injury-associated pulmonary hypertension, hypoxemia, and pulmonary edema (Schumacher, Adams et al. 1987, Kuhl, Bolds et al. 1988, Klausner, Paterson et al. 1989, Sandberg, Edberg et al. 1994, Smith, Murphy et al. 1994, Thies, Corbin et al. 1996, Quinn and Slotman 1999, Collins, Blum et al. 2001, Kobayashi, Horikami et al. 2016).

COVID-19 patients exhibit shortness of breath and low arterial blood oxygen saturation due to pulmonary edema, bronchoconstriction and reduced compliance of the lung as well as mismatching of ventilation and perfusion in alveolar gas exchange units. The cause of hypoxemia in COVID-19 is complex and not completely understood. In an animal model of lung injury following bacterial infection (i.e., sepsis), TP receptor blockade with ifetroban ameliorated systemic and pulmonary vasoconstriction and significantly increased arterial and tissue oxygenation compared with septic controls. (Quinn and Slotman 1999) A similar mitigation of hypoxemia with ifetroban may be seen in COVID-19 patients.

Isoprostanes (e.g., 8-iso-PGF2α and 8-iso-PGE2) are similar in structure to prostaglandins and also activate TP receptors (Acquaviva, Vecchio et al. 2013); however, they are produced non-enzymatically, by a pathway different from PGH2 and TxA2, following attack by oxygen-derived free radicals on phospholipids containing an esterified arachidonate moiety. The free isoprostane is released from the oxidized phospholipid by phospholipase A2. Free isoprostanes are TP receptor activators produced by mechanisms independent of cyclooxygenase and TxA synthase and are, therefore, insensitive to non-steroidal anti-inflammatory drugs and TxA synthase inhibitors. Isoprostanes are of particular interest because their synthesis is triggered by oxidative stress, their TP-receptor dependent effects are blocked by ifetroban and other TP receptor antagonists, and they are released in patients with acute lung injury or ARDS. (Carpenter, Price et al. 1998; Nanji, Liong et al. 2013; West, Voss et al. 2016)

Many COVID-19 patients develop pulmonary fibrosis, especially if they survive following mechanical ventilation and intensive care. Long-term symptoms of COVID-19 are like those caused by idiopathic pulmonary fibrosis including cough, dyspnea, and fatigue. At this time, there is COVID-19 autopsy evidence of diffuse alveolar damage (DAD) progressing to fibrosis. The authors conclude, “While we observed fibrosing DAD in fatal cases, whether or not surviving patients are at risk for developing pulmonary fibrosis and the frequency of this complication will require further clinical and radiological follow-up studies.” (Li, Wu et al. 2021)

The pathogenesis of pulmonary fibrosis has been modeled and found to be triggered by generation of free isoprostanes. 8-Iso-PGF2α activates TP receptors which leads to activation of latent TGFβ, a known mediator of fibroproliferative disorders. In the bleomycin model of pulmonary fibrosis, ifetroban blocked development of fibrosis. (Suzuki, Kropski et al. 2021) In convalescent COVID-19 patients, prevention and treatment of COVID-19-related pulmonary fibrosis is expected to become a public health problem, and effective treatment will employ a clinically effective dose regimen of TP receptor antagonist, such as ifetroban.

The most recognized biological effect of TxA2 and TP receptor activation is platelet-dependent thrombosis. Ifetroban and other TP receptor antagonists block TxA2-mediated thrombosis. Chronic hypoxia in mice produced pulmonary hypertension and pulmonary intravascular thrombosis, both of which were potentiated in COX-2 knock-out mice and prevented by treatment with ifetroban. (Cathcart, Tamosiuniene et al. 2008). In patients hospitalized with COVID-19 in a large New York City health system, thrombotic events occurred in 16.0%. Among 829 COVID-19 ICU patients, 29.4% had a thrombotic event (13.6% venous and 18.6% arterial). Among 2,505 COVID-19 non-ICU patients, 11.5% had a thrombotic event (3.6% venous and 8.4% arterial). Rates of thrombotic events in patients with COVID-19 were substantially higher than with other lung injury hospitalizations (5.9% prevalence of thrombotic events during the 2009 influenza pandemic). (Bilaloglu, Aphinyanaphongs et al. 2020) In addition to platelet-mediated thrombosis, blood coagulation initiated by expression of tissue factor on endothelial cells and monocytes can be triggered by TxA2 and TP receptor activation. (Bode, Mackman 2014) Thus, strong TP receptor signaling in pulmonary venules, platelets, monocytes and endothelial cells in the SARS-CoV-2 damaged pulmonary circulation creates an unusual prothrombotic state that is mitigated by TP receptor blockade, especially with an effective dose regimen of ifetroban.

In accordance with the present invention, it is believed that increased isoprostane signaling through the TP receptor contributes to pulmonary fibrosis in COVID-19, and thus treatment with ifetroban, an orally active TP receptor antagonist, will halt the progression of pulmonary fibrosis, improve lung function tests, and enable more complete recovery from COVID-19.

The term “TP receptor antagonist” as used herein refers to a compound that inhibits the expression or activity of a TP receptor by at least or at least about 30%, 50%, 60%, 75%, 90%, 95%, 96%, 97%, 98%, 99%, or 100% in a standard bioassay or in vivo or ex vivo when used in a therapeutically effective dose. In certain embodiments, a TP receptor antagonist inhibits binding of TxA2 to the receptor. TP receptor antagonists include competitive antagonists (i.e., antagonists that compete with an agonist for receptor occupancy) and noncompetitive antagonists. TP receptor antagonists include antibodies to the receptor. The antibodies may be monoclonal. They may be human or humanized antibodies. TP receptor antagonists may be molecules that prevent expression of the receptor with silencing RNA (i.e., siRNA) technology. TP receptor antagonists also include TxA synthase inhibitors that have both TP receptor antagonist activity and TxA synthase inhibitor activity.

TP Receptor Antagonist

The discovery and development of TP receptor antagonists has been an objective of many pharmaceutical companies for approximately 40 years. Certain individual compounds identified by these companies, either with or without concomitant TxA2 synthase inhibitory activity, include ifetroban (SQ34451; BMS-180291; Bristol-Myers Squibb), SQ29548 (BMS), SQ28668 (BMS), SQ30741 (BMS), AA-2414 (Abbott), R68070 (Janssen), BAY u 3405 (Bayer), picotamide (Sandoz), terbogrel (BI),L670596 (Merck), L655240 (Merck), ICI-192605 (Zeneca), ICI-185282 (Zeneca),ICI-159995 (Zeneca), SKF-88046 (Smith-Kline), EP-092 (U. Edinburgh), NTP-42 (ATXA), S-1452 (Shionogi), GR32191B (Glaxo), and S-18886 (Servier). Preclinical pharmacology has established that this class of compounds has effective antithrombotic activity obtained by inhibition of the prostaglandin endoperoxide and TxA2 pathway. These compounds also prevent vasoconstriction induced by TxA2 and other eicosanoids including certain isoprostanes that act on the TP receptor within vascular beds, and thus may be beneficial for use in preventing and/or treating pulmonary hypertension, fibroprolifereative disorders, hepatorenal syndrome and/or hepatic encephalopathy.

Suitable TP receptor antagonists for use in the present invention may include, for example, but are not limited to small molecules such as ifetroban {BMS; [1S-(1α,2α,3α,4α)]-2-[[3-[4-[(pentylamino)carbonyl]-2-oxazolyl]-7-oxabicyclo[2.2.1]hept-2-yl]methyl]benzenepropanoic acid; or IUPAC nomenclature: 3-[2-[[(1S,2R,3S,4R)-3-[4-(pentylcarbamoyl)-1,3-oxazol-2-yl]-7-oxabicyclo[2.2.1]heptan-2-yl]methyl]phenyl]propanoic acid}, as well as others described in U.S. Patent Application Publication No. 2009/0012115, the disclosure of which is hereby incorporated by reference in its entirety.

Additional TP receptor antagonists suitable for use herein are also described in U.S. Pat. No. 4,839,384 (Ogletree); U.S. Pat. No. 5,066,480 (Ogletree, et al.); U.S. Pat. No. 5,100,889 (Misra, et al.); U.S. Pat. No. 5,312,818 (Rubin, et al.); U.S. Pat. No. 5,399,725 (Poss, et al.); and U.S. Pat. No. 6,509,348 (Ogletree), the disclosures of which are hereby incorporated by reference in their entireties.

These may include, but are not limited to:
Interphenylene 7-oxabicyclo-heptyl substituted heterocyclic amide prostaglandin analogs as disclosed in U.S. Pat. No. 5,100,889, including:
[1S-(1α,2α,3α,4α)]-2-[[3-[4-[[(4-cyclohexylbutyl) amino]carbonyl]-2-oxazolyl]-7-oxabicyclo[2.2.1]hept-2-yl]methyl]benzenepropanoic acid (SQ 33,961), or esters or salts thereof;
[1S-(1α,2α,3α,4α)]-2-[[3-[4[[[(4-chloro-phenyl)-butyl] amino]carbonyl]-2-oxazolyl]-7-oxabicyclo[2.2.1]hept-2-yl]methyl]benzenepropanoic acid or esters, or salts thereof;
[1S-(1α,2α,3α,4α)]-2-[[3-[4-[[(4-cyclohexylbutyl)-amino]carbonyl]-2-oxazolyl]-7-oxabicyclo]2.2.1 ]hept-2-yl] benzene acetic acid, or esters or salts thereof;
[1S-(1α,2α,3α,4α)]-2-[[3-[4-[[(4-cyclohexyl-butyl) amino]carbonyl]-2-oxazolyl]-7-oxabicyclo[2.2.1]hept-2-yl]methyl]phenoxy]acetic acid, or esters or salts thereof;
[1S-(1α,2α,3α,4α)]-2-[3-[4-[[(7, 7-dimethyloctyl)-amino]carbonyl]-2-oxazolyl]-7-oxabicyclo[2.2.1]hept-2-yl]-methyl]benzenepropanoic acid, or esters or salts thereof;
7-oxabicycloheptyl substituted heterocyclic amide prostaglandin analogs as disclosed in U.S. Pat. No. 5,100,889, issued Mar. 31, 1992, including:
[1S-(1α,2α (Z), 3α,4α)]-6-[3-[4-[[(4-cyclohexylbutyl)amino]-carbonyl]-2-oxazolyl]-7-oxabicyclo[2.2.1]hept-2-yl]-4-hexenoic acid, or esters or salts thereof;
[1S-(1α,2α (Z), 3α,4α)]-6-[3-4-[[(4-cyclohexyl-butyl)amino]carbonyl]-2-thiazolyl]-7-oxabicyclo[2.2.1]hept-2-yl]-4-hexenoic acid, or esters or salts thereof;
[1S-(1α,2α (Z), 3α,4α)]-6-[3-[4-[[(4-cyclohexyl-butyl)methylamino]carbonyl]-2-oxazolyl]-7-oxabicyclo-[2.2.1]hept-2-yl]-4-hexenoic acid, or esters or salts thereof;
[1S-(1α,2α (Z), 3α,4α)]-6-[3-[4-[(1-pyrrolidinyl)-carbonyl]-2-oxazolyl]-7-oxabicyclo[2.2.1]hept-2-yl]-4-hexenoic acid, or esters or salts thereof;
[1S-(1α,2α (Z), 3α,4α)]-6-[3-[4-[(cyclohexyl-amino)-carbonyl]-2-oxazolyl]-7-oxabicyclo[2.2.1]hept-2-yl-4-hexenoic acid or esters or salts thereof;
1S-(1α,2α (Z), 3α,4α)]-6-[3-[4-[[(2-cyclohexyl-ethyl)amino]carbonyl]-2-oxazolyl]-7-oxabicyclo[2.2.1]hept-2-yl]-4-hexenoic acid, or esters or salts thereof;
[1S-(1α,2α (Z), 3α,4α)]-6-[3-[4-[[[2-(4-chloro-phenyl)ethyl]amino]carbonyl]-2-oxazolyl]-7-oxabicyclo-[2.2.1]hept-2-yl]-4-hexenoic acid, or esters or salts thereof;
[1S-(1α,2α (Z), 3α,4α)]-6-[3-[4-[[(4-chlorophenyl)amino]carbonyl]-2-oxazolyl]-7-oxabicyclo[2.2.1]hept-2-yl]-4-hexenoic acid, or esters or salts thereof;
[1S-(1α,2α (Z), 3α,4α)]-6-[3-[4-[[[4-(4-chloro-phenyl)butyl]amino]carbonyl]-2-oxazolyl]-7-oxabicyclo-[2.2.1] hept-2-yl]-4-hexenoic acid, or esters or salts thereof;
[1S-(1α,2α (Z), 3α,4α)]-6-[3-4-[[-(6-cyclohexyl-hexyl)amino]carbonyl]-2-oxazolyl]-7-oxabicyclo[2.2.1]hept-2-yl]-4-hexenoic acid, or esters, or salts thereof;
[1S-(1α,2α (Z), 3α,4α)]-6-[3-[4-[[(6-cyclohexyl-hexyl)amino]carbonyl]-2-oxazolyl]-7-oxabicyclo[2.2.1]hept-2-yl]-4-hexenoic acid, or esters or salts thereof;
[1S-(1α,2α (Z), 3α,4α)]-6-[3[4-[(propylamino)-carbonyl]-2-oxazolyl]-7-oxabicyclo[2.2.1]hept-2-yl]-4-hexenoic acid, or esters or salts thereof;
[1S-(1α,2α (Z), 3α,4α)]-6-[3-[4-[[(4-butylphenyl)amino]carbonyl]-2-oxazolyl]-7-oxabicyclo[2.2.1]hept-2-yl]-4-hexenoic acid, or esters or salts thereof;
[1S-(1α,2α (Z), 3α,4α)]-6-[3-[4-[(2,3-dihydro-1H-indol-1-yl)carbonyl]-2-oxazolyl]-7-oxabicyclo(2.2.1]hept-2-yl]-4-hexenoic acid, or esters or salts thereof;
[1S-(1α,2α (Z), 3α,4α)]-6-[3-[4-[[(4-cyclohexyl-butyl)amino]carbonyl]-2-oxazolyl]-7-oxabicyclo[2.2.1]hept-2-yl]-N-(phenylsulfonyl)-4-hexenamide;
[1S-[1α,2α (Z), 3α,4α)]]-6-[3-[4-[[(4-cyclohexyl-butyl)amino]carbonyl]-2-oxazolyl]-N-(methyl sulfonyl)- 7-oxabicyclo[2-2.1]hept-2-yl]-4-hexenamide;
[1S-(1α,2α (Z), 3α,4α)]-7-[3[4-[[(4-cyclohexyl-butyl)amino]carbonyl]-2-oxazolyl]-7-oxabicyclo(2.2.1]hept-2-yl]-5-heptenoic acid, or esters or salts thereof;
[1S-(1α,2α (Z), 3α,4α)]-6-[3-[4-[[(4-cyclohexyl-butyl)amino]carbonyl]-1H-imidazol-2-yl]-7-oxabicyclo-[2.2.1]hept-2-yl]-4-hexenoic acid or esters or salts thereof;
[1 S-[1α,2α,3α,4α]-6-[3-[4-[[(7, 7-dimethyloctyl)amino]carbonyl]-2-oxazolyl]-7-oxabicyclo[2.2.1]hept-2-yl]-4-hexenoic acid, or esters or salts thereof;
[1S-(1α,2α (E), 3α,4α)]-6-[3-[4-[[(4-cyclohexyl-butyl)amino]carbonyl]-2-oxazolyl]-7-oxabicyclo[2.2.1]hept-2-yl]-4-hexenoic acid;
1 S-(1α,2α,3α,4α)-3-[4-[[(4-(cyclohexylbutyl)-amino]carbonyl]-2-oxazolyl]-7-oxabicyclo[2.2.1]heptane-2-hexanoic acid or esters or salts thereof;
[1S-(1α,2α (Z), 3α,4α)]-6-[3-[4-[[(4-cyclohexyl-butyl)amino]carbonyl]-2-oxazolyl]-7-oxabicyclo-[2.2.1]hept-2-yl]-4-hexenoic acid, or esters or salts thereof;
7-oxabicycloheptane and 7-oxabicycloheptene compounds disclosed in U.S. Pat. No. 4,537,981 to Snitman et al, the disclosure of which is hereby incorporated by reference in its entirety, such as:
[1S-(1α,2α (Z), 3α (1E,3S*,4R*),4α)]]-7-[3-(3-hydroxy-4-phenyl-1-pentenyl)-7-oxabicyclo[2.2.1]hept-2-yl]-5-heptenoic acid (SQ 29,548);
the 7-oxabicycloheptane substituted aminoprostaglandin analogs disclosed in U.S. Pat. No. 4,416,896 to Nakane et al, the disclosure of which is hereby incorporated by reference in its entirety, such as:
[1S-(1α,2α (Z), 3α,4α)]-7-[3-[[2-(phenylamino)carbonyl]-hydrazino]methyl]-7-oxabicyclo[2.2.1]hept-2-yl]-5-heptenoic acid;
the 7-oxabicycloheptane substituted diamide prostaglandin analogs disclosed in U.S. Pat. No. 4,663,336 to Nakane et al, the disclosure of which is hereby incorporated by reference in its entirety, such as:
[1S-[1α,2α (Z), 3α,4α]]-7-[3-[[[[(1 oxoheptyl)amino]acetyl]amino]methyl]-7-oxabicyclo[2.2.1]-hept-2-yl]-5-heptenoic acid and the corresponding tetrazole, and
[1S-[1α,2α (Z), 3α,4α]]-7-[3-[[[[(4-cyclohexyl-1-oxobutyl)-amino]acetyl]amino]methyl]-7-oxabicyclo]2.2.1]hept-2-yl]-5-heptenoic acid;
7-oxabicycloheptane imidazole prostaglandin analogs as disclosed in U.S. Pat. No. 4,977,174, the disclosure of which is hereby incorporated by reference in its entirety, such as:
[1S-[1α,2α (Z), 3α,4α]]-6-[3-[[4-(4-cyclohexyl-1-hydroxybutyl)-1 H-imidazole-1-yl]methyl]-7-oxabicyclo[2.2.1]hept-2-yl]-4-hexenoic acid or its methyl ester;
[1S-[1α,2α (Z), 3α,4α]]-6-3-[[4-(3-cyclohexyl-propyl)-1H-imidazol-1-yl]methyl]-7-oxabicyclo[2.2.1]hept-2-yl]-4-hexenoic acid or its methyl ester;
[1S-[1α,2α (Z), 3α,4α]]-6-[3-[[4-cyclohexyl-1-oxobutyl)-1H-imidazol-1-yl]methyl]-7-oxabicyclo[2.2.1]hept-2-yl]-4-hexenoic acid or its methyl ester;
1S-[1α,2α (Z), 3α,4α]]-6[3-(1H-imidazol-1-ylmethyl)-7-oxabicyclo[2.2.1]hept-2-yl]-4-hexenoic acid or its methyl ester; or
[1S-[1α,2α (Z), 3α,4α]]-6-[3-[[4-[[(4-cyclohexyl-butyl)amino]carbonyl]-1H-imidazol-1-yl]methyl-7-oxabicyclo-[2.2.1]-hept-2-yl]-4-hexenoic acid, or its methyl ester;
The phenoxyalkyl carboxylic acids disclosed in U.S. Pat. No. 4,258,058 to Witte et al, the disclosure of which is hereby incorporated by reference in its entirety, including:

BM 13.177: 2[4-[2-(benzenesulfonamido)ethyl]phenoxy]acetic acid (sulotroban, Boehringer Mannheim);

The sulphonamidophenyl carboxylic acids disclosed in U.S. Pat. No. 4,443,477 to Witte et al, the disclosure of which is hereby incorporated by reference in its entirety, including:

BM 13.505: 2-[4-[2-[(4-chlorophenyl)sulfonylamino]ethyl]phenyl]acetic acid (daltroban, Boehringer Mannheim);

The arylthioalkylphenyl carboxylic acids disclosed in U.S. Pat. No. 4,752,616, the disclosure of which is hereby incorporated by reference in its entirety, including 4-(3-((4-chlorophenyl)sulfonyl)propyl)benzene acetic acid.

Other examples of thromboxane A2 receptor antagonists suitable for use herein include, but are not limited to:
R68070: 5-[(E)-[pyridin-3-yl-[3-(trifluoromethyl)phenyl]methylidene]amino]oxypentanoic acid (ridogrel, Janssen),
L670596: (—)6,8-difluoro-9-p-methylsulfonylbenzyl-1,2,3,4-tetrahydrocarbazol-1-yl-acetic acid (Merck),
L655240: 3-[1-[(4-chlorophenyl)methyl]-5-fluoro-3-methyl-2-indolyl]-2,2-dimethylpropanoic acid (Merck-Frosst),
ICI-192,605: 4(Z)-6-[(2,4,5-cis)2-chloropheny 1)-4-(2-hydroxyphenyl)-1,3-dioxan yl]hexenoic acid (ICI, Zeneca),
ICI-185282: (Z)-7-[(2S,4S,5R)-4-(2-hydroxyphenyl)-2-(trifluoromethyl)-1,3-dioxan-5-yl]hept-5-enoic acid (ICI, Zeneca),
ICI-159995: 5(Z)-7-[2,2-dimethyl-4-phenyl-1,3-dioxan-cis-5-yl]heptanoic acid (ICI, Zeneca),
SKF-88046: N,N′-bis[7-(3-chlorobenzeneaminosulfonyl)-1,2,3,4-tetrahydro-isoquinolyl]disulfonylimide (Smith Kline),
EP-092: (Z,2-endo-3-oxo)-7-(3-acetyl-2-bicyclo[2.2.1]heptyl-5-hepta-3Z-enoic acid, 4-phenyl-thiosemicarbazone (Univ. Edinburgh),
AH-23848: (E)-7-[2-morpholin-4-yl-3-oxo-5-[(4-phenylphenyl)methoxy]cyclopentyl]hept-4-enoic acid (Glaxo),
GR-32,191B: (Z)-7-[(1R,2R,3 S,5S)-3-hydroxy-5-[(4-phenylphenyl)methoxy]-2-piperidin-1-ylcyclopentyl]hept-4-enoic acid (vapiprost; Glaxo),
BAY u 3405: 3-[[(4-fluorophenyl)-sulfonyl]amino]-1,2,3,4-tetrahydro-9H-carbazole-9-propanoic acid; (ramatroban; Bayer),
ONO-3708: ((1S,2S,3S,5R)-3-((R)-2-cyclopentyl-2-hydroxyacetamido)-6,6-dimethylbicyclo[3.1.1]heptan-2-yl)hept-5-enoic acid (ONO),
S-1452: (Z)-7-[(1R,2S,3S,4S)-3-(benzenesulfonamido)-2-bicyclo[2.2.1]heptanyl]hept-5-enoic acid (domitroban, Anboxan®, Shionogi),
S-18886: 3-[(6R)-6-[(4-chlorophenyl)sulfonylamino]-2-methyl-5,6,7,8-tetrahydronaphthalen-1-yl]propanoic acid (terutroban, Servier),
AA-2414: 7-phenyl-7-(2,4,5-trimethyl-3,6-dioxocyclohexa-1,4-dien-1-yl)heptanoic acid (seratrodast, Abbott),
NTP-42: 1-tert-butyl-3-[5-cyano-2-[3-[4-(difluoromethoxy)phenyl]phenoxy]phenyl]sulfonylurea (ATXA Therapeutics),
Picotamide: 4-methoxy-1-N,3-N-bis(pyridin-3-ylmethyl)benzene-1,3-dicarboxamide (Sandoz),
Linotroban: 5(2-(phenylsulfonylamino)ethyl)-thienyloxy-acetic acid (Nycomed),

The preferred TP receptor antagonist of the present invention is ifetroban or any pharmaceutically acceptable salts thereof. In certain preferred embodiments the preferred TP receptor antagonist is ifetroban sodium (known chemically as 3-[2-[[(1S,2R,3S,4R)-3-[4-(pentylcarbamoyl)-1,3-oxazol-2-yl]-7-oxabicyclo[2.2.1]heptan-2-yl]methyl]phenyl]propanoic acid, monosodium salt.

Methods of Treatment

In certain embodiments of the present invention there is provided a method of treating and/or ameliorating COVID-19 in a patient or patient population by administration of a therapeutically effective amount of a TP receptor antagonist to a patient(s) in need thereof. The administration of a therapeutically effective amount of a TP receptor antagonist may be accomplished via any therapeutically useful route of administration, including but not limited to orally, intranasally, by inhalation, rectally, vaginally, sublingually, buccally, parenterally, or transdermally.

In certain preferred embodiments, the TP receptor antagonist is administered orally. In certain further embodiments, the TP receptor antagonist is administered by parenteral injection. In certain further embodiments, the TP receptor antagonist is administered by inhalation directly to the lungs. In certain preferred embodiments, the plasma concentrations of TP receptor antagonists range from about 0.1 ng/mL to about 10,000 ng/mL. Preferably, the plasma concentration of TP receptor antagonists range from about 1 ng/mL to about 1,000 ng/mL. When the TP receptor antagonist is ifetroban, the desired plasma concentration for treatment of COVID-19 in certain embodiments should be greater than about 10 ng/mL (ifetroban free acid). Some therapeutic effects of TP receptor antagonist, e.g., ifetroban, may be seen at concentrations of greater than about 1 ng/mL. The dose administered should be adjusted according to age, weight and condition of the patient, fed or fasted state, as well as the route of administration, dosage form and regimen and the desired result.

In order to obtain the desired plasma concentration of TP receptor antagonists for the treatment of COVID-19 patients, daily doses of the TP receptor antagonists preferably range from about 0.1 mg to about 5,000 mg. In certain preferred embodiments, the TP receptor antagonist is administered on a chronic basis. Daily doses may range from about 1 mg to about 1,000 mg; about 10 mg to about 1,000 mg; about 50 mg to about 250 mg; about 100 mg to about 500 mg; about 200 mg to about 500 mg; about 300 mg to about 500 mg; or from about 400 mg to about 500 mg per day. In certain preferred embodiments where the animal is a human patient, the therapeutically effective amount is from about 50 mg to about 2,000 mg per day, or from about 10 mg to 250 mg per day, or from about 200 mg to about 1,000 mg per day, and certain embodiments more preferably from about 50 to about 500 mg per day, or from about 100 mg to about 500 mg per day.

The daily dose may be administered in divided doses or in one bolus or unit dose or in multiple dosages administered concurrently. In this regard, the ifetroban may be administered orally, intranasally, by inhalation, rectally, vaginally, sublingually, buccally, parenterally, or transdermally. In certain preferred embodiments, the pharmaceutical composition described above, the therapeutically effective amount is from about 10 mg to about 300 mg ifetroban (or a pharmaceutically acceptable salt thereof) per day. In certain preferred embodiments, the therapeutically effective amount is from about 50 to about 250 mg per day, and in certain embodiments from about 150 mg to about 350 mg per day will produce therapeutically effective plasma levels of ifetroban free acid for the treatment COVID-19. In certain preferred embodiments, a daily dose of ifetroban sodium from about 10 mg to about 250 mg (ifetroban free acid amounts) will produce therapeutically effective plasma levels of ifetroban free acid for the treatment of COVID-19.

Preferably, the therapeutically effective plasma concentration of TP receptor antagonists ranges from about 1 ng/mL to about 1,000 ng/mL for the treatment of COVID-19. When the TP receptor antagonist is ifetroban, the desired plasma concentration for providing an inhibitory effect versus TP receptor activation, and thus a reduction of platelet activation should be greater than about 10 ng/mL (ifetroban free acid). Some inhibitory effects of TP receptor antagonist, e.g., ifetroban, may be seen at concentrations of greater than about 1 ng/mL.

The dose administered must be carefully adjusted according to age, weight and condition of the patient, as well as the route of administration, dosage form and regimen and the desired result. However, in order to obtain the desired plasma concentration of TP receptor antagonists, daily doses of the TP receptor antagonists ranging from about 1 mg to about 5000 mg should be administered. Preferably, the daily dose of TP receptor antagonists ranges from about 1 mg to about 1000 mg; about 10 mg to about 1000 mg; about 50 mg to about 500 mg; about 100 mg to about 500 mg; about 200 mg to about 500 mg; about 300 mg to about 500 mg; and about 400 mg to about 500 mg per day. In certain preferred embodiments, a daily dose of ifetroban sodium from about 10 mg to about 250 mg (ifetroban free acid amounts) will produce effective plasma levels of ifetroban free acid.

Pharmaceutical Compositions

The TP receptor antagonists of the present invention may be administered by any pharmaceutically effective route. For example, the TP receptor antagonists may be formulated in a manner such that they can be administered orally, intranasally, by inhalation, rectally, vaginally, sublingually, buccally, parenterally, or transdermally, and, thus, be formulated accordingly.

In certain embodiments, the TP receptor antagonists may be formulated in a pharmaceutically acceptable oral dosage form. Oral dosage forms may include, but are not limited to, oral solid dosage forms and oral liquid dosage forms. Oral solid dosage forms may include, but are not limited to, tablets, capsules, caplets, powders, pellets, multiparticulates, beads, spheres and any combinations thereof. These oral solid dosage forms may be formulated as immediate release, controlled release, sustained (extended) release or modified release formulations. The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

Depending on the desired release profile, the oral solid dosage forms of the present invention may contain a suitable amount of controlled-release agents, extended-release agents, or modified-release agents.

Oral liquid dosage forms include, but are not limited to, solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol and combinations thereof. In certain embodiments of the present invention, the TP receptor antagonists may be formulated into a dosage form suitable for parenteral use. For example, the dosage form may be a lyophilized powder, a solution, suspension (e.g., depot suspension). In other embodiments, the TP receptor antagonists may be formulated into a topical dosage form such as, but not limited to, a patch, a gel, a paste, a cream, an emulsion, liniment, balm, lotion, and ointment.

### DETAILED DESCRIPTION OF THE PREFERRED EMBODIMENTS

The following examples are not meant to be limiting and represent certain embodiments of the present invention.

### Example I

In this example, ifetroban sodium tablets are prepared with the following ingredients listed in Table 1:

TABLE 1

| Ingredients | Percent by weight |
|---|---|
| Na salt of Ifetroban | 35 |
| Mannitol | 50 |
| Microcrystalline Cellulose | 8.0 |
| Crospovidone | 3.0 |
| Magnesium Oxide | 2.0 |
| Magnesium Stearate | 1.5 |
| Colloidal Silica | 0.3 |

The sodium salt of ifetroban, magnesium oxide, mannitol, microcrystalline cellulose, and crospovidone is mixed together for about 2 to about 10 minutes employing a suitable mixer. The resulting mixture is passed through a #12 to #40 mesh size screen. Thereafter, magnesium stearate and colloidal silica are added and mixing is continued for about 1 to about 3 minutes. The resulting homogeneous mixture is then compressed into tablets each containing 35 mg, ifetroban sodium salt.

### Example II

In this example, 1,000 tablets each containing 400 mg of Ifetroban sodium are produced from the following ingredients listed in Table 2:

TABLE 2

| Ingredients | Amount | |
|---|---|---|
| Na salt of Ifetroban | 400 | gm |
| Corn Starch | 50 | g |
| Gelatin | 7.5 | g |
| Microcrystalline Cellulose (Avicel) | 25 | g |
| Magnesium Stearate | 2.5 | g |

### Example III

An injectable solution of ifetroban sodium is prepared for intravenous use with the following ingredients listed in Table 3:

TABLE 3

| Ingredients | Amount | |
|---|---|---|
| Ifetroban Sodium | 2500 | mg |
| Methyl Paraben | 5 | mg |
| Propyl Paraben | 1 | mg |
| Sodium Chloride | 25,000 | mg |
| Water for injection, q.s. | 5 | liters |

The sodium salt of ifetroban, preservatives and sodium chloride are dissolved in 3 liters of water for injection and then the volume is brought up to 5 liters. The solution is filtered through a sterile filter and aseptically filled into pre-sterilized vials which are then closed with pre-sterilized rubber closures. Each vial contains a concentration of 75 mg of active ingredient per 150 mL of solution.

### REFERENCES

Ackermann, M., S. E. Verleden, M. Kuehnel, A. Haverich, T. Welte, F. Laenger, A. Vanstapel, C. Werlein, H. Stark, A. Tzankov, W. W. Li, V. W. Li, S. J. Mentzer and D. Jonigk (2020). “Pulmonary Vascular Endothelialitis, Thrombosis, and Angiogenesis in Covid-19.” N Engl J Med 383(2): 120-128.
Acquaviva, A., D. Vecchio, B. Arezzini, M. Comporti and C. Gardi (2013). “Signaling pathways involved in isoprostane-mediated fibrogenic effects in rat hepatic stellate cells.” Free Radic Biol Med 65: 201-207.
Barrett, T. J., A. H. Lee, Y. Xia, L. H. Lin, M. Black, P. Cotzia, J. Hochman and J. S. Berger (2020). “Platelet and Vascular Biomarkers Associate With Thrombosis and Death in Coronavirus Disease.” Circ Res 127(7): 945-947.
Begley, C. J., M. L. Ogletree, B. O. Meyrick and K. L. Brigham (1984). “Modification of pulmonary responses to endotoxemia in awake sheep by steroidal and nonsteroidal anti-inflammatory agents.” Am Rev Respir Dis 130(6): 1140-1146.
Bertolino, F., J. P. Valentin, M. Maffre, A. M. Bessac and G. W. John (1995). “TxA2 receptor activation elicits organ-specific increases in microvascular permeability in the rat.” Am J Physiol 268(2 Pt 2): R366-374.
Bilaloglu, S., Y. Aphinyanaphongs, S. Jones, E. Iturrate, J. Hochman and J. S. Berger (2020). “Thrombosis in Hospitalized Patients With COVID-19 in a New York City Health System.” JAMA 324(8): 799-801.
Bode, M. and N. Mackman (2014). “Regulation of tissue factor gene expression in monocytes and endothelial cells: Thromboxane A2 as a new player.” Vascul Pharmacol 62(2): 57-62.
Brigham, K. L., C. J. Begley, G. R. Bernard, A. A. Hutchison, J. E. Loyd, W. D. Lucht, B. Meyrick, J. H. Newman, M. E. Niedermeyer, M. L. Ogletree and et al. (1983). “Septicemia and lung injury.” Clin Lab Med 3(4): 719-744.
Caravita, S., C. Baratto, F. Di Marco, A. Calabrese, G. Balestrieri, F. Russo, A. Faini, D. Soranna, G. B. Perego, L. P. Badano, L. Grazioli, F. L. Lorini, G. Parati and M. Senni (2020). “Haemodynamic characteristics of COVID-19 patients with acute respiratory distress syndrome requiring mechanical ventilation. An invasive assessment using right heart catheterization.” Eur J Heart Fail 22(12): 2228-2237.
Carpenter, C. T., P. V. Price and B. W. Christman (1998). “Exhaled breath condensate isoprostanes are elevated in patients with acute lung injury or ARDS.” Chest 114(6): 1653-1659.
Cathcart, M. C., R. Tamosiuniene, G. Chen, T. G. Neilan, A. Bradford, K. J. O'Byrne, D. J. Fitzgerald and G. P. Pidgeon (2008). “Cyclooxygenase-2-linked attenuation of hypoxia-induced pulmonary hypertension and intravascular thrombosis.” J Pharmacol Exp Ther 326(1): 51-58.
Chow, J. H., A. K. Khanna, S. Kethireddy, D. Yamane, A. Levine, A. M. Jackson, M. T. McCurdy, A. Tabatabai, G. Kumar, P. Park, I. Benjenk, J. Menaker, N. Ahmed, E. Glidewell, E. Presutto, S. Cain, N. Haridasa, W. Field, J. G. Fowler, D. Trinh, K. N. Johnson, A. Kaur, A. Lee, K. Sebastian, A. Ulrich, S. Pena, R. Carpenter, S. Sudhakar, P. Uppal, B. T. Fedeles, A. Sachs, L. Dahbour, W. Teeter, K. Tanaka, S. M. Galvagno, D. L. Herr, T. M. Scalea and M. A. Mazzeffi (2021). “Aspirin Use Is Associated With Decreased Mechanical Ventilation, Intensive Care Unit Admission, and In-Hospital Mortality in Hospitalized Patients With Coronavirus Disease 2019.” Anesth Analg 132(4): 930-941.
Collins, B. J., M. G. Blum, R. E. Parker, A. C. Chang, K. S. Blair, G. L. Zorn, 3rd, B. W. Christman and R. N. Pierson, 3rd (2001). “Thromboxane mediates pulmonary hypertension and lung inflammation during hyperacute lung rejection.” J Appl Physiol (1985) 90(6): 2257-2268.
Ganter, C. C., S. M. Jakob and J. Takala (2006). “Pulmonary capillary pressure. A review.” Minerva Anestesiol 72(1-2): 21-36.
Gu, S. X., T. Tyagi, K. Jain, V. W. Gu, S. H. Lee, J. M. Hwa, J. M. Kwan, D. S. Krause, A. I. Lee, S. Halene, K. A. Martin, H. J. Chun and J. Hwa (2021). “Thrombocytopathy and endotheliopathy: crucial contributors to COVID-19 thromboinflammation.” Nat Rev Cardiol 18(3): 194-209.
Gupta, A., K. Kalantar-Zadeh and S. T. Reddy (2020). “Ramatroban as a Novel Immunotherapy for COVID-19.” J Mol Genet Med 14(3).
Hasan, S. S., T. Capstick, R. Ahmed, C. S. Kow, F. Mazhar, H. A. Merchant and S. T. R. Zaidi (2020). “Mortality in COVID-19 patients with acute respiratory distress syndrome and corticosteroids use: a systematic review and meta-analysis.” Expert Rev Respir Med 14(11): 1149-1163.
Klausner, J. M., I. S. Paterson, G. Goldman, L. Kobzik, C. R. Valeri, D. Shepro and H. B. Hechtman (1989). “Thromboxane A2 mediates increased pulmonary microvascular permeability following limb ischemia.” Circ Res 64(6): 1178-1189.
Kobayashi, K., D. Horikami, K. Omori, T. Nakamura, A. Yamazaki, S. Maeda and T. Murata (2016). “Thromboxane A2 exacerbates acute lung injury via promoting edema formation.” Sci Rep 6: 32109.
Kuhl, P. G., J. M. Bolds, J. E. Loyd, J. R. Snapper and G. A. FitzGerald (1988). “Thromboxane receptor-mediated bronchial and hemodynamic responses in ovine endotoxemia.” Am J Physiol 254(2 Pt 2): R310-319.
Li, Y., J. Wu, S. Wang, X. Li, J. Zhou, B. Huang, D. Luo, Q. Cao, Y. Chen, S. Chen, L. Ma, L. Peng, H. Pan, W. D. Travis and X. Nie (2021). “Progression to fibrosing diffuse alveolar damage in a series of 30 minimally invasive autopsies with COVID-19 pneumonia in Wuhan, China.” Histopathology 78(4): 542-555.
Liu, Q., N. Huang, A. Li, Y. Zhou, L. Liang, X. Song, Z. Yang and X. Zhou (2021). “Effect of low-dose aspirin on mortality and viral duration of the hospitalized adults with COVID-19.” Medicine (Baltimore) 100(6): e24544.
Majidi, S., J. T. Fifi, T. R. Ladner, J. Lara-Reyna, K. A. Yaeger, B. Yim, N. Dangayach, T. J. Oxley, T. Shigematsu, B. R. Kummer, L. K. Stein, J. Weinberger, M. G. Fara, R. De Leacy, M. S. Dhamoon, S. Tuhrim and J. Mocco (2020). “Emergent Large Vessel Occlusion Stroke During New York City's COVID-19 Outbreak: Clinical Characteristics and Paraclinical Findings.” Stroke 51(9): 2656-2663.
Marshall, M. (2020). “The lasting misery of coronavirus long-haulers.” Nature 585(7825): 339-341.
McDonald, L. T. (2021). “Healing after COVID-19: are survivors at risk for pulmonary fibrosis?” Am J Physiol Lung Cell Mol Physiol 320(2): L257-L265.
Nakahata, N. (2008). “Thromboxane A2: physiology/pathophysiology, cellular signal transduction and pharmacology.” Pharmacol Ther 118(1): 18-35.
Nanji, A. A., E. C. Liong, J. Xiao and G. L. Tipoe (2013). “Thromboxane inhibitors attenuate inflammatory and fibrotic changes in rat liver despite continued ethanol administrations.” Alcohol Clin Exp Res 37(1): 31-39.
Ogletree, M. L., C. J. Begley, G. A. King and K. L. Brigham (1986). “Influence of steroidal and nonsteroidal anti-inflammatory agents on the accumulation of arachidonic acid metabolites in plasma and lung lymph after endotoxemia in awake sheep. Measurements of prostacyclin and thromboxane metabolites and 12-HETE.” Am Rev Respir Dis 133(1): 55-61.
Ojo, A. S., S. A. Balogun, 0. T. Williams and O. S. Ojo (2020). “Pulmonary Fibrosis in COVID-19 Survivors: Predictive Factors and Risk Reduction Strategies.” Pulm Med 2020: 6175964.
Pulley, J. M., R. N. Jerome, M. L. Ogletree, G. R. Bernard, R. R. Lavieri, N. M. Zaleski, C. C. Hong, J. K. Shirey-Rice, C. L. Arteaga, I. A. Mayer, K. J. Holroyd and R. S. Cook (2018). “Motivation for Launching a Cancer Metastasis Inhibition (CMI) Program.” Target Oncol 13(1): 61-68.
Quinn, J. V. and G. J. Slotman (1999). “Platelet-activating factor and arachidonic acid metabolites mediate tumor necrosis factor and eicosanoid kinetics and cardiopulmonary dysfunction during bacteremic shock.” Crit Care Med 27(11): 2485-2494.
Sandberg, K., K. E. Edberg, W. Fish, R. A. Parker, C. Hellerqvist and H. Sundell (1994). “Thromboxane receptor blockade (SQ 29,548) in group B streptococcal toxin challenge in young lambs.” Pediatr Res 35(5): 571-579.
Schumacher, W. A., H. D. Adams and M. L. Ogletree (1987). “Effect of the thromboxane A2-receptor antagonists, SQ 29,548 and SQ 28,668, on the pulmonary hypertensive response to endotoxemia in swine.” Pharmacology 34(6): 301-308.
Schuster, D. P., Kozlowski, J., Brimioulle, S. (2001). Effect of thromboxane receptor blockade on pulmonary capillary hypertension in acute lung injury. American Thoracic Society.
Smith, W. J., M. P. Murphy, R. F. Appleyard, R. J. Rizzo, L. Aklog, R. G. Laurence and L. H. Cohn (1994). “Prevention of complement-induced pulmonary hypertension and improvement of right ventricular function by selective thromboxane receptor antagonism.” J Thorac Cardiovasc Surg 107(3): 800-806.
Suzuki, T. Kropski, J. Chen, J. Carrier, E. Chen, X. Sherrill, T. Polosukhin, V. Han, W. Rathinasabapathy, A. Gutor, S. Gulleman, P. Banovich, N. Tanjore, H. Freeman, M. Tada, Y. Young, L. Gokey, J. Blackwell, T. West, J. (2021). The F2-Isoprostane:Thromboxane-prostanoid receptor signaling axis drives persistent fibroblast activation in pulmonary fibrosis. (Submitted for publication)
Tarantino, E., P. Amadio, I. Squellerio, B. Porro, L. Sandrini, L. Turnu, V. Cavalca, E. Tremoli and S. S. Barbieri (2016). “Role of thromboxane-dependent platelet activation in venous thrombosis: Aspirin effects in mouse model.” Pharmacol Res 107: 415-425. Thies, S. D., R. S. Corbin, C. D. Goff, 0. A. Binns, S. A. Buchanan, K. S. Shockey, H. F. Frierson, Jr., J. S. Young, C. G. Tribble and I. L. Kron (1996). “Thromboxane receptor blockade improves oxygenation in an experimental model of acute lung injury.” Ann Thorac Surg 61(5): 1453-1457.
Wakerlin, G. E., Jr., J. C. Finn, L. C. Siegel, G. V. Benson, T. F. Flavin and R. G. Pearl (1995). “Pulmonary capillary pressure measurement from pulmonary artery occlusion pressure decay profile analysis in sheep.” Anesth Analg 81(1): 17-23.
Werfel, T. A., D. J. Hicks, B. Rahman, W. E. Bindeman, M. T. Duvernay, J. G. Maeng, H. Hamm, R. R. Lavieri, M. M. Joly, J. M. Pulley, D. L. Elion, D. M. Brantley-Sieders and R. S. Cook (2020). “Repurposing of a thromboxane receptor inhibitor based on a novel role in metastasis identified by Phenome Wide Association Study.” Mol Cancer Ther.
West, J. D., C. L. Galindo, K. Kim, J. J. Shin, J. B. Atkinson, I. Macias-Perez, L. Pavliv, B. C. Knollmann, J. H. Soslow, L. W. Markham and E. J. Carrier (2019). “Antagonism of the Thromboxane-Prostanoid Receptor as a Potential Therapy for Cardiomyopathy of Muscular Dystrophy.” J Am Heart Assoc 8(21): e011902.
West, J. D., B. M. Voss, L. Pavliv, M. de Caestecker, A. R. Hemnes and E. J. Carrier (2016). “Antagonism of the thromboxane-prostanoid receptor is cardioprotective against right ventricular pressure overload.” Pulm Circ 6(2): 211-223.
Yoshimura, K., M. L. Tod, K. G. Pier and L. J. Rubin (1989). “Role of venoconstriction in thromboxane-induced pulmonary hypertension and edema in lambs.” J Appl Physiol (1985) 66(2): 929-935.

While the present invention has been described in its preferred embodiments, it is to be understood that the words which have been used are words of description rather than of limitation and that changes may be made within the purview of the appended claims without departing from the true scope and spirit of the invention in its broader aspects. Rather, various modifications may be made in the details within the scope and range of equivalents of the claims and without departing from the spirit of the invention. The inventor further requires that the scope accorded their claims be in accordance with the broadest possible construction available under the law as it exists on the date of filing hereof (and of the application from which this application obtains priority, if any) and that no narrowing of the scope of the appended claims be allowed due to subsequent changes in the law, as such a narrowing would constitute an ex post facto adjudication, and a taking without due process or just compensation.

1. A method of treating COVID-19, comprising administering a therapeutically effective amount of a thromboxane A2 receptor antagonist to a patient suffering from SARS-CoV-2 infection and/or COVID-19 symptoms.

2. The method of claim 1, wherein the COVID-19 patient is treated by administering the thromboxane A2 antagonist in an outpatient, inpatient and/or convalescent phase.

3. The method of claim 2, wherein the thromboxane A2 receptor antagonist is selected from the group of 3-[2-[[(1S,2R,3S,4R)-3-[4-(pentylcarbamoyl)-1,3-oxazol-2-yl]-7-oxabicyclo[2.2.1]heptan-2-yl]methyl]phenyl]propanoic acid (Ifetroban), or one, or a mixture of more than one pharmaceutically acceptable salts thereof.

4. The method of claim 2, wherein the thromboxane A2 receptor antagonist is 3-[2-[[(1S,2R,3 S,4R)-3-[4-(pentylcarbamoyl)-1,3-oxazol-2-yl]-7-oxabicyclo[2.2.1]heptan-2-yl]methyl]phenyl]propanoic acid, monosodium salt (Ifetroban Sodium).

5. The method of claim 1, wherein the thromboxane A2 receptor antagonist is administered either orally, intranasally, by inhalation, rectally, vaginally, sublingually, buccally, parenterally, or transdermally, or any combination thereof.

6. The method of claim 2, wherein the thromboxane A2 receptor antagonist is administered prophylactically to prevent development of respiratory failure in an outpatient or inpatient stage.

7. The method of claim 2, wherein the thromboxane A2 receptor antagonist is administered to treat and prevent progression of pulmonary fibrosis in the patient.

8. The method of claim 2, wherein the therapeutically effective amount is from about 10 mg to about 1,500 mg, per day.

9. The method of claim 2, wherein the therapeutically effective amount is from about 10 mg to about 500 mg per day, and the thromboxane A2 receptor antagonist is administered parenterally.

10. The method of claim 2, wherein the therapeutically effective amount is from about 50 mg to about 1,500 mg per day, and the thromboxane A2 receptor antagonist is administered orally.

11. A method of treating pulmonary dysfunction in a human patient suffering from COVID-19, comprising chronically administering a therapeutically effective amount of a thromboxane A2 receptor antagonist to the human patient.

12. The method of claim 11, wherein the therapeutically effective amount is from about 10 mg to about 1,500 mg, per day.

13. The method of claim 11, wherein the thromboxane A2 receptor antagonist is selected from the group of 3-[2-[[(1S,2R,3S,4R)-3-[4-(pentylcarbamoyl)-1,3-oxazol-2-yl]-7-oxabicyclo[2.2.1]heptan-2-yl]methyl]phenyl]propanoic acid (Ifetroban), or one, or a mixture of more than one pharmaceutically acceptable salts thereof.

14. The method of claim 11, wherein the thromboxane A2 receptor antagonist is 3-[2-[[(1 S,2R,3 S,4R)-3-[4-(pentylcarbamoyl)-1,3-oxazol-2-yl]-7-oxabicyclo[2.2.1]heptan-2-yl]methyl]phenyl]propanoic acid, monosodium salt (Ifetroban Sodium).

15. The method of claim 13, wherein the therapeutically effective amount is from about 50 mg to about 250 mg per day, and the ifetroban is administered orally.

16. The method of claim 11, wherein the pulmonary dysfunction is pulmonary capillary hypertension.

17. The method of claim 11, wherein the pulmonary dysfunction is pulmonary edema.

18. The method of claim 11, wherein the pulmonary dysfunction is pulmonary fibrosis.

19. The method of claim 11, wherein the pulmonary dysfunction is thrombotic microangiopathy.

20. The method of claim 2, wherein the COVID-19 patient is a person less than 60 years of age, lacking a history of thrombosis, and is administered the thromboxane A2 antagonist to prevent or treat large vessel thrombotic angiopathy.
